(19) United States
(12) Patent Application Publication
(10) Pub. No.: US 2026/0108044 A1
(43) Pub. Date: Apr. 23, 2026

(54) COSMETIC CONTAINER TUBE WITH CHARGING ELECTRODES

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/269,095

(22) Filed: Jul. 15, 2025

(30) Foreign Application Priority Data
Oct. 18, 2024 (CN) 2024225309719

(51) Int. Cl. A45D 34/04; A61N 1/32; A45D 34/00

(52) U.S. Cl. CPC A45D 34/04; A61N 1/328; A45D 2034/002

(57) ABSTRACT

Embodiments of the present invention disclose a cosmetic container comprising a container body and a detachable applicator head. The container body includes a dispensing end and a closed end, and is provided with a first electrical interface at the closed end and a second electrical interface at the dispensing end, electrically connected via internal conductive components. The applicator head includes a mating electrical interface, a rechargeable battery, and a stimulation element, such as a phototherapy lamp. When mounted, the mating electrical interface aligns with the second electrical interface of the container body, allowing the battery to be charged through the first electrical interface provided on the container. The applicator head further includes a coated application surface with a liquid outlet channel and a light-transmitting portion, enabling simultaneous cosmetic dispensing and light-based therapy to improve user convenience and skincare effectiveness.

### TECHNICAL FIELD

The present invention relates to the technical field of cosmetic packaging, and more particularly to a cosmetic container equipped with charging electrodes and an integrated stimulation element, which allows for the combination of skincare element storage, electric connectivity, and therapeutic functionality within a single modular system.

### BACKGROUND

Cosmetic containers have long served as essential packaging solutions for storing and dispensing various skincare and beauty products, such as creams, serums, lotions, and gels. Traditionally, these containers have taken the form of simple bottles, jars, or tubes made from plastic, glass, or metal. Their primary function has been to preserve product integrity, ensure hygienic dispensing, and provide a user-friendly means of application. Most conventional cosmetic containers featured a passive delivery mechanism, requiring the user to manually extract the product and apply it to the skin using fingers, applicator sticks, or external tools.

Over time, advancements in skincare science and consumer expectations led to the introduction of cosmetic containers with integrated applicators, such as roller balls, sponge tips, and brush heads. Some later designs incorporated powered elements such as vibration, heating, or light therapy elements to enhance skincare routines. These powered applicator heads were often attached to the opening of the container and were driven by batteries embedded in the applicator head’s body. While these hybrid solutions attempted to combine storage, application, and therapy, they introduced new complications in terms of size, usability, and design complexity.

A major limitation of these conventional systems was the need to include a charging interface or port directly on the applicator head itself in order to recharge the battery. This requirement increased the applicator head's size and weight, making it bulky and less compatible with compact cosmetic containers. The conventional designs typically required the removal of the applicator head, or the applicator head had to be turned upside down, or a change in the angle to facilitate charging, which often led to issues of liquid leakage. However, the present container-based charging system eliminates this inconvenience by enabling the application device to be charged while the applicator head remains attached to the container or without changing the angle. Moreover, incorporating a charging port often compromised aesthetic appeal, introduced sealing challenges, and limited design flexibility. As a result, smaller or travel-sized skincare containers were often excluded from such value-added features, and consumers had to compromise between portability and advanced skincare treatment.

Additionally, the removal of the applicator head, inversion of the device, or adjustment of its angle to facilitate charging often used to results in liquid leakage. The container-based charging of the cosmetic container eliminates these issues by allowing the device to be charged seamlessly while the applicator head remains securely attached. Moreover, the charger may be configured as either a wired or a wireless connector or a charging coil, adapted to accommodate the tube and enable efficient charging without requiring disassembly or leakage problems.

The present utility model addresses these shortcomings by introducing an innovative cosmetic container structure in which the charging interface is relocated from the applicator head to the container body. The container body is equipped with an first electrical interface (positioned at the closed end) and a second electrical interface (positioned at a liquid dispensing end), which are electrically connected through internal conductive paths. The applicator head, which remains detachably mountable at the container’s dispensing end, contains the battery, a stimulation element (e.g., phototherapy lamp or microcurrent electrode), and a mating electrical interface for interface with the container’s output conductive parts.

This structural reconfiguration allows the applicator head to remain lightweight, compact, and free of bulky charging ports, significantly enhancing usability, especially for small or travel-friendly cosmetic containers. It also improves sealing reliability and industrial design possibilities, while still supporting powered therapeutic functions. The result is a modular, space-efficient, and functionally integrated cosmetic container that bridges the gap between traditional packaging and modern, technology-enhanced skincare application. This invention thus fills a significant void in the cosmetic market by offering enhanced convenience, functionality, and aesthetic value in one streamlined system.

### OBJECTS OF THE INVENTION

Some of the objects of the invention are as follows:

An object of the present invention is to provide a cosmetic container with an integrated electrical charging system that eliminates the need for a charging port on the applicator head, thereby reducing its size and improving usability.

Another object of the present invention is to provide a cosmetic container wherein a detachable applicator head is powered through internal conductive connections between a first electrical interface at the container’s closed end and a second electrical interface at the dispensing end.

Another object of the invention is to provide a compact cosmetic applicator head that includes a battery-powered stimulation element, such as a phototherapy lamp(Light Emitting Diodes (LEDs), or lasers elements), heating elements, cooling elements, vibratory elements, electrodes, etc., for enhanced skincare treatment.

Another object of the present invention is to provide an application surface integrated with both a liquid outlet channel and a light-transmitting portion, enabling simultaneous product application and light-based therapy.

Another object of the present invention is to provide a modular applicator head structure adaptable to various container designs using an interchangeable adapter interface.

### SUMMARY OF THE INVENTION

According to a first aspect of the present invention, a cosmetic container system is provided. The cosmetic container system comprising: a container body having a dispensing end and an opposite end, the container body comprising: a first electrical interface disposed on or within the container body; a conductive pathway extending between the first electrical interface and a second electrical interface located at or near the dispensing end; an applicator head configured to detachably attach to the dispensing end of the container body, the applicator head comprising: a mating electrical interface configured to establish electrical connection with the second electrical interface of the container body; and a power storage element electrically connected to the mating electrical interface.

In one embodiment of the invention, the power storage element comprises a rechargeable battery.

In one embodiment of the invention, the conductive pathway comprises at least one conductive trace or wire embedded within a wall of the container body.

In one embodiment of the invention, the first electrical interface is exposed at the outer surface of the opposite end of the container body for connection to an external charging device.

In one embodiment of the invention, the second electrical interface comprises a pair of conductive contacts arranged circumferentially around a fluid outlet at the dispensing end.

In one embodiment of the invention, the mating electrical interface comprises one or more spring-loaded pins or pogo pins configured to maintain electrical contact with the second electrical interface.

In one embodiment of the invention, the applicator head further comprises one or more stimulation elements electrically connected to the power storage element and configured to provide a cosmetic or therapeutic effect.

In one embodiment of the invention, the one or more stimulation elements comprise a light source configured to emit light through a transparent or translucent portion of the applicator head.

In one embodiment of the invention, the container body is configured to store a cosmetic substance, wherein the container body comprises an outer shell and a replaceable inner container removably disposed within the outer shell.

In one embodiment of the invention, the container comprises an opening on the dispensing end configured to fill a cosmetic substance in the container body in a retrofillable arrangement.

According to a second aspect of the present invention, a cosmetic container system is provided. The cosmetic container system comprising: a container body having a dispensing end and an opposite end, the container body comprising: a first electrical interface disposed on or within the container body; a conductive pathway extending between the first electrical interface and a second electrical interface located at or near the dispensing end; an applicator head configured to detachably attach to the dispensing end of the container body, the applicator head comprising: a mating electrical interface comprising at least one resilient contact member configured to establish electrical contact with the second electrical interface of the container body; and a power storage element electrically connected to the mating electrical interface.

In one embodiment of the invention, the first electrical interface is exposed on an external surface of the opposite end of the container body and is configured to receive power from an external power source.

In one embodiment of the invention, the second electrical interface comprises a pair of conductive electrodes positioned around a fluid outlet at the dispensing end of the container body.

In one embodiment of the invention, the resilient contact member of the applicator head comprises a pair of spring pins, each spring pin having a conductive abutment portion configured to resiliently contact a corresponding conductive electrode of the second electrical interface.

In one embodiment of the invention, the resilient contact member is biased to maintain continuous electrical contact with the second electrical interface during user operation or movement.

According to a third aspect of the present invention, a method of operating a cosmetic container system comprising a container body and a detachable applicator head is provided. The method comprising the steps of: supplying an electrical charge to a first electrical interface disposed on or within the container body; conducting the electrical charge from the first electrical interface through at least one conductive pathway extending along an interior portion of the container body; delivering the electrical charge from the conductive pathway to a second electrical interface positioned at a dispensing end of the container body; establishing electrical contact between the second electrical interface and one or more resilient contact members of a mating electrical interface disposed on the applicator head; and transferring the electrical charge through the resilient contact members to a power storage element disposed within the applicator head.

In one embodiment of the invention, the conductive pathway comprises at least one metal strip affixed along an inner surface of the container body and electrically insulated from a cosmetic substance stored within the container body.

In one embodiment of the invention, the method further comprises the step of using the electrical charge stored in the power storage element to operate a stimulation element integrated within the applicator head.

In one embodiment of the invention, the stimulation element performs at least one of: phototherapy, micro-current, piezoelectric massage, ultrasonic wave, thermal stimulation, or vibration therapy.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term "several" means more than one, unless otherwise specified.

In the context of the specification, the term "LED" refers to one or more light-emitting diode (LED) elements that are electrically connected and configured to emit light of specific wavelengths (including visible to IR wavelengths) suitable for therapeutic purposes. The LED element may include drive circuitry, heat dissipation structures, and optical elements such as lenses or diffusers to control light distribution.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source”, etc., refers to a source emitting coherent laser light, or light-emitting diodes (“LEDs”). The term “light therapy” refers to light generated from any of the sources, such as lasers, LED sources, or Super Luminous Diodes (“SLD”), or Organic light-emitting diodes (OLED).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user. The electrodes are placed at or near nerves where the pain is located or at certain known trigger points.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a cosmetic container, in accordance with an embodiment of the present invention.

FIG. 2 illustrates a schematic diagram of a detached container, marked with a point ‘A’ of the cosmetic container, in accordance with an embodiment of the present invention.

FIG. 3 shows an enlarged view of point ‘A’ illustrated in FIG. 2, in accordance with an embodiment of the present invention.

FIG. 4 illustrates a schematic diagram of the structure of a first and a second electrical interface on a corresponding conductive pathway, a metal strip of the cosmetic container, in accordance with an embodiment of the present invention.

FIG. 5 illustrates a bottom view of an applicator head of the cosmetic container, in accordance with an embodiment of the present invention.

FIG. 6 illustrates a cross-sectional view of the applicator head of the cosmetic container, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The embodiments of the present invention provide an applicator head and a cosmetic container system with charging electrodes that seamlessly integrate a skincare storage reservoir with advanced therapeutic and electrical functionalities. In one embodiment, the system comprises a container body and a detachable applicator head, together forming a modular unit suitable for delivering cosmetic products alongside stimulation-based skincare treatments. The container body includes a dispensing end equipped with a second electrical interface and a closed end equipped with a first electrical interface configured for external charging. A conductive pathway, such as a metal strip, extends through the container wall from the closed end to the dispensing end, connecting this first electrical interface to a second electrical interface positioned near the dispensing end. The applicator head, attachable to the dispensing end through an addapter, houses a power storage element, such as a rechargeable battery, electrically connected via a mating electrical interface incorporating spring-loaded pins. The applicator head further includes at least one stimulation element, such as a phototherapy lamp, vibration motor, microcurrent electrode, ultrasonic wave element or heating element, and an application surface with a liquid outlet channel and a light-transmitting portion.

In another embodiment of the present invention, the cosmetic container system operates through a coordinated charging and stimulation sequence facilitated by the first and second electrical interfaces. The cosmetic container system includes a container body having a first electrical interface disposed at or within the closed end, and a second electrical interface disposed at or near the dispensing end. These two interfaces are electrically connected via an embedded conductive pathway, such as one or more metal strips, aligned along the surface of the container wall and electrically insulated from the stored cosmetic substance.

In an embodiment, the method of operation of the cosmetic container system begins with supplying an electrical charge to the first electrical interface, which is accessible on the outer surface of the closed end of the container body. The first electrical interface is connected to an external charging source, either through direct physical contacts, magnetic connectors, or wireless charging coils. Upon activation of the power source, electrical current flows through the conductive pathway within the container body, reaching the second electrical interface positioned near the dispensing end.

When the applicator head is mounted onto the dispensing end of the container body, a mating electrical interface integrated into the applicator head, comprising a pair of spring-loaded pins or pogo pins, comes into direct contact with the second electrical interface. The spring-loaded pins are mechanically biased to maintain continuous electrical contact, even under movement or varying orientations during user operation, ensuring stable and uninterrupted power transfer from the container body to the applicator head.

The transferred electrical charge is delivered to a power storage element, such as a rechargeable battery, embedded within the applicator head. The stored power is then utilized to activate one or more stimulation elements housed in the applicator head, including but not limited to phototherapy lamps (e.g., LEDs), heating or cooling elements, vibration elements, ultrasonic wave elements, or microcurrent electrodes.

Once the cosmetic liquid is dispensed through the aligned liquid outlet channel disposed on the applicator head and connected internally to the liquid outlet tube, and applied to the user’s skin, the stimulation element can be triggered, either manually via a switch or automatically through integrated sensors or timers. The stimulation element then delivers its therapeutic effect, enhancing product absorption, skin toning, blood circulation, or targeted treatment of skin conditions. The cosmetic container system allows the user to operate the cosmetic container and stimulation applicator without detaching any components or adjusting the orientation of the container, offering leak-free charging, reliable electrical engagement, and the ability to deliver both skincare products and treatment simultaneously in a user-friendly and compact form factor.

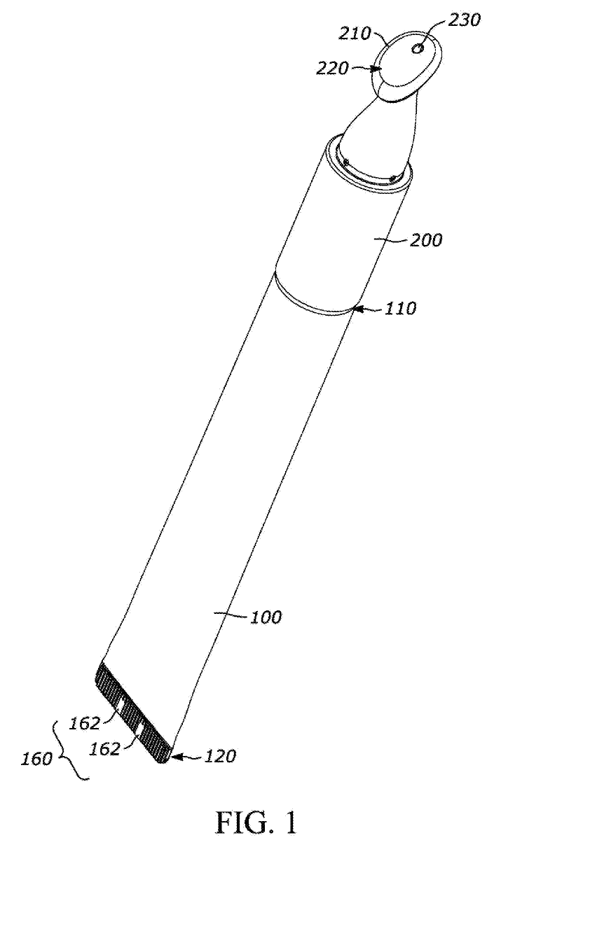

Referring to FIG. 1, a perspective view of the phototherapy cosmetic applicator is provided. The phototherapy cosmetic applicator comprises a container body 100, an applicator head 200. The container body 100 includes a dispensing end 110 and a closed end 120 that is equipped with a first electrical interface 160, exposed externally on the container body 100. The applicator head 200 is detachably mounted on the dispensing end 110 and accommodates components like a battery, main control board, and a stimulation element. The stimulation element is selected from a group consisting of, but not limited to, a phototherapy lamp with one or more LEDs or a laser, a piezoelectric element, a vibration element, a heating element, a cooling element, a thermal element, or a micro-current element.

In addition, the applicator head 200 includes an application surface 210 and a liquid outlet channel 230. One end of the liquid outlet channel 230 passes through the application surface 210, while the other end is in fluid communication with the interior of the container body 100, specifically connected to the liquid outlet 132. During use, the cosmetic product stored in the container body 100 flows through the liquid outlet channel 230 and is dispensed via the application surface 210, allowing the user to directly apply the cosmetic to the skin with ease and precision.

In some embodiments, the application surface 210 may be coated with a biocompatible material selected to enhance both user comfort and functional performance. The coating can include a metallic layer, such as gold, stainless steel, or chrome, to provide a smooth, hygienic, and corrosion-resistant finish, particularly beneficial if the application surface also serves as a microcurrent electrode.

Alternatively, the application surface 210 may be coated with a soft elastomeric or silicone-based material to deliver a gentle tactile feel suitable for sensitive skin, improving comfort during cosmetic application. The application surface 210 may also incorporate an antibacterial or antifouling coating to maintain hygiene and reduce microbial buildup over repeated uses. When integrated with phototherapy or electrical stimulation, the coating may be conductive, enabling efficient delivery of therapeutic energy while maintaining skin safety and performance reliability.

The application surface 210 is designed to come into direct contact with the user’s skin during use and is further provided with a light-transmitting portion 220, aligned with the phototherapy lamp to transmit the light of the phototherapy lamp to the user’s skin and provide the benefits of the light therapy to the user’s skin.

This configuration enables the stimulation element 600 to be activated after the application of a skin care product, thereby facilitating deeper absorption of the cosmetic into the skin through any of the stimulation element 600, including light, micro-current, heat, cold, ultrasonic wave, or vibration stimulation. The light-transmitting portion 220 may be formed from a transparent material or may include a through-hole, allowing light emitted by the stimulation element 600 or phototherapy lamp to effectively pass through and reach the user’s skin. In a similar way, the through-hole of light-transmitting portion 220 allows the microcurrent electrodes to effectively pass through and reach the user’s skin.

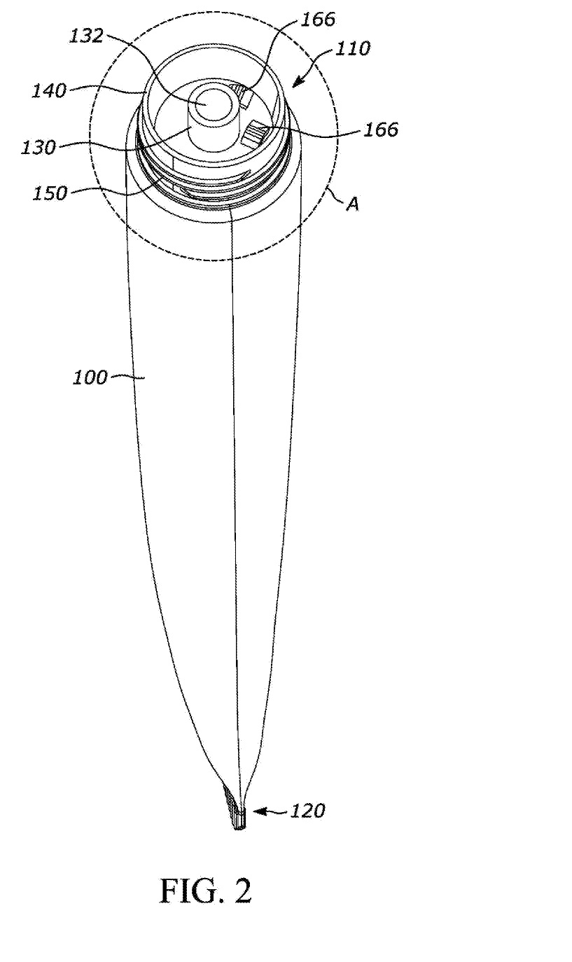

FIG. 2 illustrates a schematic diagram of a container body 100 detached from the applicator body. The container body 100 is shown independently, without the attachment of the applicator head 200, thereby clearly revealing the structural features configured on the container body itself. The container body 100 comprises a dispensing end 110 and a closed end 120 located opposite to each other. The main body of the container body 100 is designed to store cosmetic liquid, such as eye cream, serum, or other skincare formulations, and serves as the primary reservoir of the container.

The dispensing end 110 is centrally equipped with a liquid outlet tube 130, which has a liquid outlet 132. The liquid outlet 132 is in fluid communication with the internal cavity of the container body 100, allowing the cosmetic fluid to flow outward when pressure is applied to the container body. Surrounding the liquid outlet tube 130 is an annular connecting portion 140, which has external threads 150 to facilitate detachable mechanical engagement with a corresponding internal thread provided on the applicator head. This threading arrangement ensures a secure fit between the container body 100 and the applicator head 200, allowing for user-friendly assembly and disassembly.

Adjacent to the annular connecting portion 140, two output conductive parts 166 of a second electrical interface 172, embedded or mounted within, near or outside the dispensing end 110. The output conductive parts 166 are spaced circumferentially between the annular connecting portion 140 and the liquid outlet tube 130. Each output conductive part 166 may include a guide surface 168, which is inclined relative to the vertical axis of the container body 100, thereby promoting guided engagement with corresponding conductive abutment parts of a mating electrical interface 300 during the attachment of the applicator head.

The closed end 120 of the container body 100 includes the first electrical interface 160, which has a pair of input conductive parts 162. The two input conductive parts 162 are exposed externally and are configured for wired or wireless electrical connection with an external power source or charger. The input conductive parts 162 are structurally integrated into the container body, either via moulded embedding or adhesive attachment, and are internally connected through a conductive pathway to the output conductive parts 166 via metal strips 174. The positioning of the input conductive parts 162 at the closed end 120 ensures that charging operations can occur independently without interfering with the use of the applicator head 200 on the dispensing end 110.

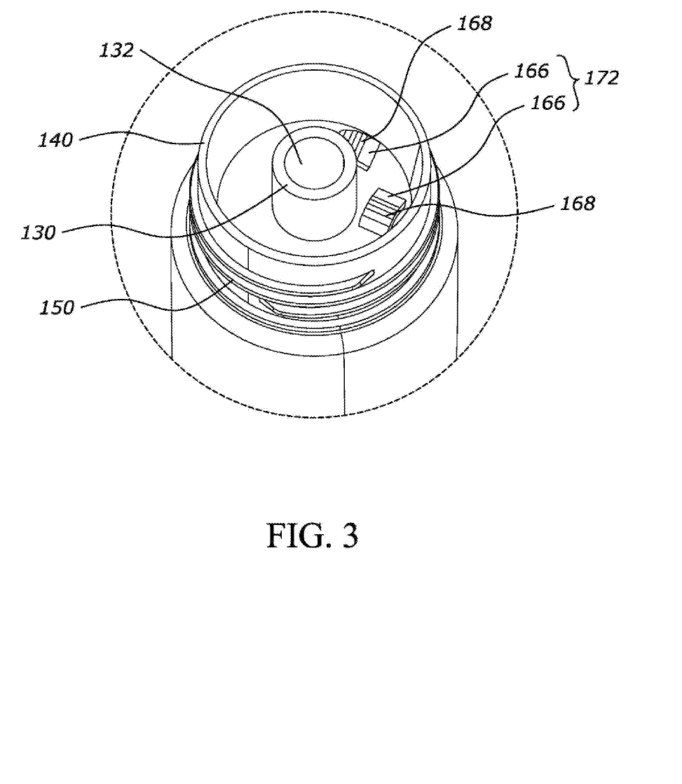

FIG. 3 shows an enlarged view of point ‘A’, providing a more detailed view of the structural arrangement and electrical connectivity components located at the dispensing end 110 of the container body 100, depicting the spatial configuration and orientation of the output conductive parts 166, the guide surfaces 168, and their integration into the surrounding structures, which facilitate both electrical conductivity and guided mechanical coupling with the applicator head 200.

The dispensing end 110 of the container body 100 integrates a moulded base or grooves that accommodate the output conductive parts 166 and guide surfaces 168. The liquid outlet tube 130, centrally located at the dispensing end 110, accommodates a liquid outlet 132 for dispensing the cosmetic fluid. Surrounding the liquid outlet tube 130 is the annular connecting portion 140, which provides mechanical support for attachment to the applicator head. The external thread 150 is provided around the annular connecting portion 140, allowing for secure threaded engagement with the corresponding internal thread of the applicator head.

Positioned between the annular connecting portion 140 and the liquid outlet tube 130 are the two output conductive parts 166, which form a key part of the second electrical interface 172. These conductive parts are arranged in a circumferentially spaced configuration and are embedded or fixed within the structure of the dispensing end 110. Each output conductive part 166 features a guide surface 168, which is inclined relative to the central axis of the container body. These inclined guide surfaces serve to direct and align the conductive abutment portion, a pair of spring-loaded pins 310 of the mating electrical interface 300 of the applicator head, during attachment, ensuring precise and reliable electrical contact without the need for complex positioning mechanisms.

The inclined geometry of the guide surfaces 168 also contributes to self-centring functionality during the mounting of the applicator head. As the applicator head is rotated or pushed into position, the spring-loaded pins naturally slide along the guide surfaces 168 and come to rest in firm electrical contact with the output conductive parts 166. This not only simplifies user operation but also enhances the durability and reliability of the contact interface, hence resists dislodgement and wear during repeated assembly and disassembly cycles

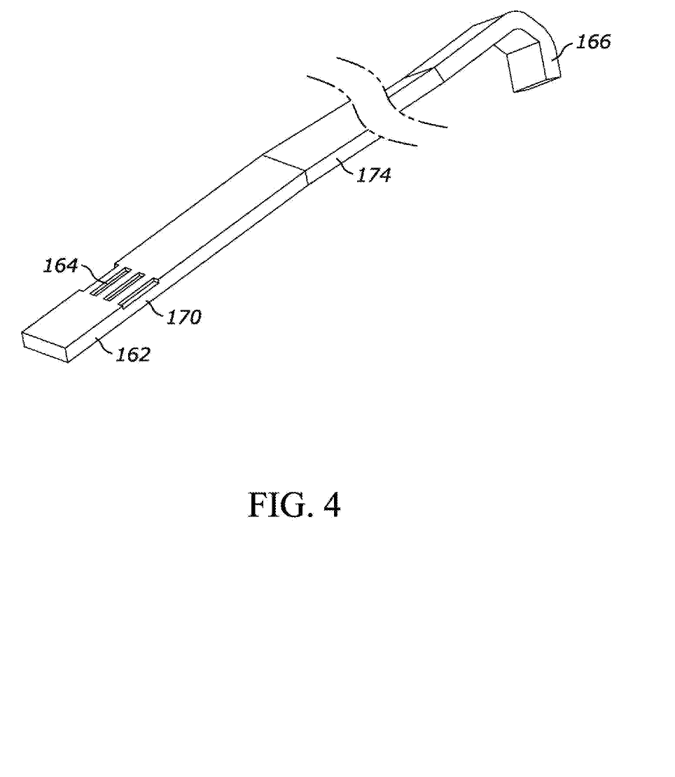

FIG. 4 illustrates a schematic diagram of the structure of the first and second electrical interfaces on a corresponding metal strip of the cosmetic container, highlighting the internal electrical pathway that connects the first electrical interface 160 at the closed end 120 of the container body 100 to the second electrical interface 172 positioned at the dispensing end 110. This internal electrical linkage is crucial for transmitting charging current from an external power source to the battery 500 mounted in the applicator head.

The input conductive part 162, located at the closed end 120 and works as a conductive pad or terminal, configured to be exposed on the external surface of the container body 100 for ease of contact with a charging device. On the other hand, the output conductive part 166 is located near the dispensing end 110, where it interfaces with the mating electrical interface 300 during assembly. Both first and second electrical interfaces are electrically connected via an elongated metal strip 174, which forms a conductive pathway and is positioned along the inner wall of the container body 100.

The metal strip 174 acts as a transmission path for electrical current. One end of the metal strip 174 is electrically joined to the input conductive part 162, while the opposite end is electrically joined to the output conductive part 166. This configuration ensures a direct, low-resistance electrical connection that extends along the vertical axis of the container body 100. In certain embodiments, two such metal strips may be arranged in parallel, one for the positive and one for the negative or ground connection, thereby completing a dual-conductor circuit from the base to the top of the container.

Further, the fixing part 170 is integrally formed or attached to the input conductive part 162 to mechanically secure it within the closed end of the container body. The fixing part 170 may feature a concave-convex structure 164, which enhances bonding and alignment when embedded into the wall or housing of the container body 100 at the closed end 120. The integration of the metal strip 174 with the help of the fixing part 170 at the closed end of the container body 100. The structural design and integration of the metal strip 174 provide mechanical stability and ensure that the conductive elements remain securely positioned during use and handling.

Furthermore, the metal strip 174 can be surface-mounted on the inner wall of the container body, with its outer surface optionally coated with an insulating layer to prevent unintentional short circuits or external contact. In other configurations, the metal strip 174 may be overmolded or embedded during the injection molding process, thereby becoming a permanent, protected part of the container’s structure. The presence of a metal strip 174 eliminates the need for additional wires or external ports within the applicator head 200, thus plays a critical role in achieving the objective of miniaturizing the applicator head by relocating the charging interface to the container body, while still maintaining reliable power delivery to the battery 500 located within the applicator head.

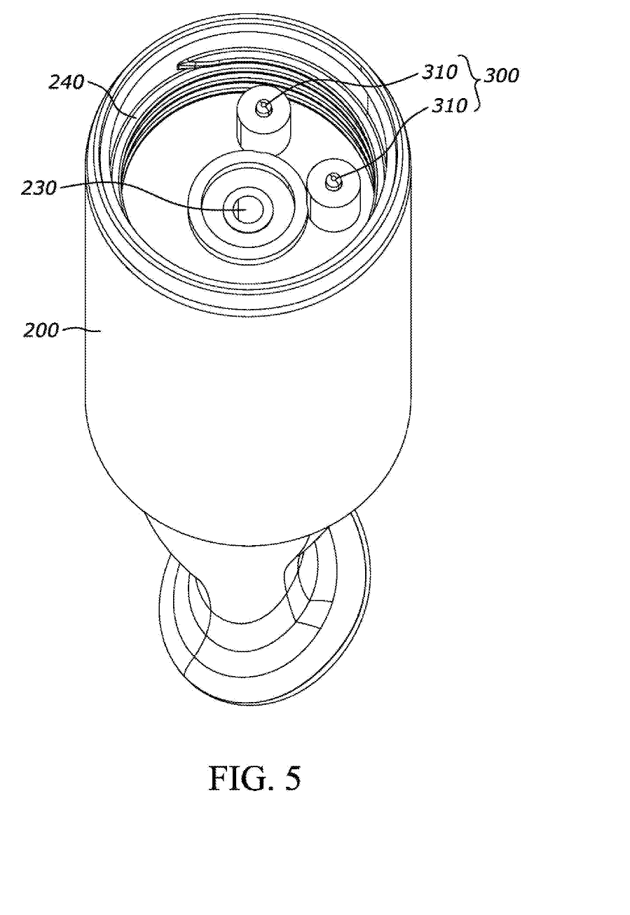

FIG. 5 illustrates a bottom view of the applicator head 200 of the cosmetic container, providing insight into the functional layout of its lower face, which interfaces directly with the container body. The applicator head 200 is designed to be detachably mounted to the dispensing end of the container and performs multiple roles, including dispensing cosmetic fluid and supporting therapeutic functions. The housing 204 of the applicator head forms a secure enclosure for internal components such as the battery and control board, while its bottom end contains the outlet and electrical contact structures that interact with the main container.

Centrally located within the applicator head 200 is the liquid outlet channel 230, which serves as a passage through which cosmetic material is dispensed from the container body. The liquid outlet channel 230 runs vertically from the interior of the container to the application surface, allowing the user to apply the cosmetic fluid directly to the skin. The liquid outlet channel 230 is precisely aligned with the container’s liquid outlet tube when the applicator head is mounted, ensuring an efficient and leak-free flow of the product. The positioning of the liquid outlet channel 230 at the centre of the applicator head 200 enables symmetrical design and optimal ergonomic use.

The internal thread 240 surrounds the lower opening of the applicator head 200, which is moulded or machined into the inner circumference of the housing 204. The internal thread 240 is configured to engage with the external thread 150 provided on the annular connecting portion 140 of the container body. The threaded engagement between parts ensures a firm and stable mechanical connection, allowing the applicator head to be screwed on or off with ease. This also enhances user convenience for recharging, cleaning, or replacing the applicator head as needed. Additionally, the applicator head and container body may engage together using alternative connection mechanisms such as snap-fit, magnetic couplings, clamps-hooks or latching systems. These configurations offer design flexibility and facilitate user-friendly attachment and removal.

Adjacent to the liquid outlet channel 230 and built into the base of the applicator head is a mating electrical interface 300, which includes a pair of spring-loaded pins 310. These spring-loaded pins 310 are designed to extend outward and align with the output conductive parts 166 on the container body 100 when the applicator head is mounted. As part of the electrical interface, the spring-loaded pins 310 maintain reliable contact pressure even when minor misalignments occur, which enables stable power delivery from the battery charging system embedded in the container body 100 to the internal components of the applicator head, the stimulation element 600 such as a phototherapy element, vibration element, heating element, cooling element, thermal element or microcurrent elements etc. The integration of mating electrical interface 300 and spring-loaded pins 310 into the bottom face of the applicator head contributes to compactness and simplifies the electrical coupling process.

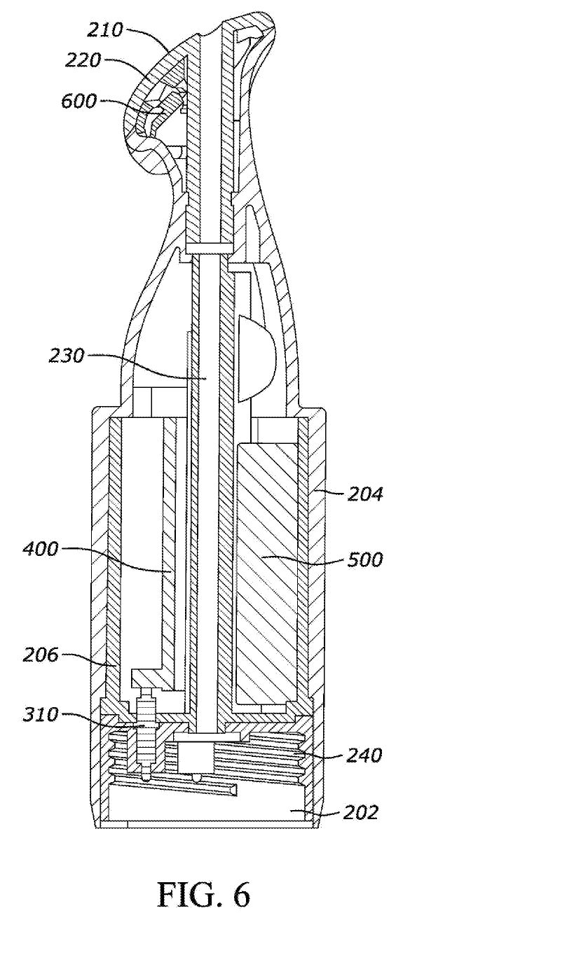

FIG. 6 illustrates a cross-sectional view of the applicator head 200 of the cosmetic container body, showing the internal structure and spatial arrangement of key components housed within the applicator head 200, providing a vertical slice from the application surface 210 to the interface that connects with the container body.

At the lower end of the applicator head 200 is the application surface 210, which forms the external face that comes into contact with the user’s skin. The application surface 210 is part of the housing 204, which encloses and supports the applicator head's internal architecture. The housing 204 encloses a battery 500, a main control board 400, and a stimulation element 600. The stimulation element may include a phototherapy element, a vibration element, a heating element, a cooling element, a thermal element, an ultrasonic wave element or a microcurrent element, etc.

The stimulation element 600 may be implemented as a single functional unit or as a combination of multiple elements within the applicator head 200, allowing the device to deliver one or more therapeutic effects simultaneously or sequentially. Such combinations may include vibration, heating, cooling, phototherapy, ultrasonic stimulation, piezoelectric and microcurrent therapy.

The application surface 210 may be treated or coated with materials that enhance both its functionality and user experience. For example, a metallic coating such as stainless steel, chrome, or gold may be applied to provide a durable, smooth, and hygienic surface, particularly useful when the application surface functions as part of a microcurrent therapy system.

Alternatively, the application surface 210 may be a soft silicone or elastomeric layer used to create a skin-friendly contact surface that ensures comfort during use, especially on sensitive areas. To promote cleanliness during repeated applications, the application surface 210 may also be coated with an antibacterial or antimicrobial treatment. In cases where the application surface is used for light or electrical therapy, a conductive coating may be employed to facilitate efficient energy transfer while ensuring safe and consistent skin contact.

Passing through the application surface 210 is the liquid outlet channel 230, which connects directly to the liquid outlet 132 of the container body. The liquid outlet channel 230 enables cosmetic liquid to flow from the internal reservoir of the container, through the applicator head, and onto the user's skin during use. The liquid may be actuated manually, such as by squeezing or pressing the container, or may be assisted by a built-in pump or similar dispensing system.

Moreover, the application surface aligns with a light-transmitting portion 220, made from a transparent or translucent material. A phototherapy lamp, one of the stimulation elements 600, is positioned directly below the light-transmitting portion 220. The stimulation element 600 is mounted on a main control board 400 that governs its operation. When powered, the lamp emits light through the light-transmitting portion 220 toward the user's skin, enabling therapeutic treatment such as LED-based absorption enhancement or anti-inflammatory care. The placement of the phototherapy lamp 600 ensures that light treatment occurs precisely where the cosmetic is applied.

The battery 500, located within the housing 204 and mounted onto a bracket 206. The battery 500 supplies power to the stimulation element 600.

The stimulation element 600 integrated within, near or on the applicator head 200 may serve different therapeutic functions depending on the desired skincare treatment. The stimulation element incorporates a phototherapy lamp, such as an LED or laser light source, configured to emit specific wavelengths of light, such as red, blue, or near-infrared, for skin benefits including acne reduction, collagen stimulation, and inflammation relief. When the stimulation element 600 is configured as a vibration element, which is coupled to an eccentric rotating mass (ERM) motor or a linear resonant actuator (LRA) to provide vibration therapy to the skin.

In an embodiment where the stimulation element 600 provides heating, it may be selected from a group including metal-based, ceramic, semiconductor, thick-film, polymer-based, composite, or combination heating elements. If cooling is desired, the stimulation element 600 may take the form of a thermoelectric cooler, such as a Peltier heat pump. For ultrasonic therapy, the stimulation element 600 may include an ultrasonic wave generation surface formed by bonding a quartz crystal to a metal plate of the application surface 210, wherein the quartz crystal emits ultrasonic waves via the piezoelectric effect. Such ultrasonic stimulation may aid in skin rejuvenation, enhance blood circulation, promote tissue repair, and support the treatment of chronic skin conditions. Additionally, the application surface 210 of the applicator head 200 may include microcurrent electrodes configured to deliver low-level electrical stimulation for improving muscle tone, enhancing product absorption, and promoting skin firmness.

An adapter 202 is positioned at the open end of the housing 204 and is configured for detachable connection with the dispensing end 110 of the container body 100. The mating electrical interface 300 is integrated into the adapter 202, allowing it to establish electrical contact with the output conductive parts of the container body. Within the adapter is the mating electrical interface 300, including spring-loaded pins 310, which are oriented to align with the output conductive parts 166 of the container body 100. These spring-loaded pins 310 ensure reliable electrical contact, allowing the internal battery to receive power from the container's integrated input-output circuit system.

This modular design allows the applicator head 200 to be easily adapted to various container configurations by simply replacing or customizing the adapter 202, thereby enhancing compatibility and manufacturing flexibility. In one specific embodiment, the adapter 202 is provided with an internal thread 240, allowing it to securely mate with a corresponding external thread on the container body 100.

The container body 100 may be made up from a variety of materials, including plastic, metal, or glass, and may take the form of any conventional or specialized cosmetic container including, but is not limited to, bottle-type containers, collapsible tubes, or hybrid forms such as dropper bottles, brush-tipped applicators with integrated tubes, or roller-ball dispensers. The design is compatible with a wide range of skincare oils, medicinal liquids, creams and serum products, ensuring broad applicability across different cosmetic formats.

Additionally, the inner container or refill unit of the bottle tube may be configured to be replaceable and supports a retrofillable arrangement, allowing users to refill the cosmetic product without replacing the entire outer tube structure. This modular configuration enhances sustainability, facilitates product replenishment, and extends the usability of the cosmetic container.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A cosmetic container system, comprising:
a container body having a dispensing end and an opposite end, the container body comprising:
a first electrical interface disposed on or within the container body;
a conductive pathway extending between the first electrical interface and a second electrical interface located at or near the dispensing end;
an applicator head configured to detachably attach to the dispensing end of the container body, the applicator head comprising:
a mating electrical interface configured to establish electrical connection with the second electrical interface of the container body; and a power storage element electrically connected to the mating electrical interface.

2. The cosmetic container system of claim 1, wherein the power storage element comprises a rechargeable battery.

3. The cosmetic container system of claim 1, wherein the conductive pathway comprises at least one conductive trace or wire embedded within a wall of the container body.

4. The cosmetic container system of claim 1, wherein the first electrical interface is exposed at an outer surface of the opposite end of the container body for connection to an external charging device.

5. The cosmetic container system of claim 1, wherein the second electrical interface comprises a pair of conductive contacts arranged circumferentially around a fluid outlet at the dispensing end.

6. The cosmetic container system of claim 1, wherein the mating electrical interface comprises one or more spring-loaded pins or pogo pins configured to maintain electrical contact with the second electrical interface.

7. The cosmetic container system of claim 1, wherein the applicator head further comprises one or more stimulation elements electrically connected to the power storage element and configured to provide a cosmetic or therapeutic effect.

8. The cosmetic container system of claim 7, wherein the one or more stimulation elements comprise a light source configured to emit light through a transparent or translucent portion of the applicator head.

9. The cosmetic container system of claim 1, wherein the container body is configured to store a cosmetic substance, and wherein the container body comprises an outer shell and a replaceable inner container removably disposed within the outer shell.

10. The cosmetic container system of claim 1, wherein the container comprises an opening on the dispensing end configured to fill a cosmetic substance in the container body in a retrofillable arrangement.

11. A cosmetic container system, comprising:
a container body having a dispensing end and an opposite end, the container body comprising:
a first electrical interface disposed on or within the container body; a conductive pathway extending between the first electrical interface and a second electrical interface located at or near the dispensing end;
an applicator head configured to detachably attach to the dispensing end of the container body, the applicator head comprising: a mating electrical interface comprising at least one resilient contact member configured to establish electrical contact with the second electrical interface of the container body; and
a power storage element electrically connected to the mating electrical interface.

12. The cosmetic container system of claim 11, wherein the first electrical interface is exposed on an external surface of the opposite end of the container body and is configured to receive power from an external power source.

13. The cosmetic container system of claim 11, wherein the second electrical interface comprises a pair of conductive electrodes positioned around a fluid outlet at the dispensing end of the container body.

14. The cosmetic container system of claim 13, wherein the resilient contact member of the applicator head comprises a pair of spring pins, each spring pin having a conductive abutment portion configured to resiliently contact a corresponding conductive electrode of the second electrical interface.

15. The cosmetic container system of claim 11 further comprising at least one stimulation element selected from a group consisting of one or more LEDs, a vibration element, a heating element, a cooling element, a thermal element, or a micro-current element.

16. The cosmetic container system of claim 11, wherein the resilient contact member is biased to maintain continuous electrical contact with the second electrical interface during user operation or movement.

17. A method of operating a cosmetic container system comprising a container body and a detachable applicator head, the method comprising:
supplying an electrical charge to a first electrical interface disposed on or within the container body;
conducting the electrical charge from the first electrical interface through at least one conductive pathway extending along an interior portion of the container body;
delivering the electrical charge from the conductive pathway to a second electrical interface positioned at a dispensing end of the container body;
establishing electrical contact between the second electrical interface and one or more resilient contact members of a mating electrical interface disposed on the applicator head; and
transferring the electrical charge through the resilient contact members to a power storage element disposed within the applicator head.

18. The method of claim 17, wherein the conductive pathway comprises at least one metal strip affixed along an inner surface of the container body and electrically insulated from a cosmetic substance stored within the container body.

19. The method of claim 17 further comprises the step of using the electrical charge stored in the power storage element to operate a stimulation element integrated within the applicator head.

20. The method of claim 19, wherein the stimulation element performs at least one of: phototherapy, micro-current, massage, thermal stimulation, or vibration therapy.
